(19) United States
(12) Patent Application Publication
(10) Pub. No.: US 2011/0230823 A1
(43) Pub. Date: Sep. 22, 2011

(54) VALVE MECHANISM FOR A SUCTION AND IRRIGATION INSTRUMENT

(75) Inventor: Jesper Schantz Simonsen, Copenhagen (DK)

(73) Assignee: Lina Medical ApS

(21) Appl. No.: 12/671,770

(22) PCT Filed: Aug. 9, 2007
(86) PCT No.: PCT/IB07/53162

§ 371 Date: Feb. 2, 2010

(51) Int. Cl. A61M 1/00

(52) U.S. Cl. 604/30; 604/246

(57) ABSTRACT

A valve mechanism for a suction or irrigation instrument, that includes a first tubular housing provided with at least one first distal conduit assembly for coupling to the instrument, and a conduit for communicating with a suction or irrigation source. The conduit assembly includes at least first and second proximal conduits, and a first piston movably arranged in the first tubular housing between first and second positions. The first piston has a first through-opening intersecting the longitudinal axis of the first piston, wherein upon movement of the first piston into the first position the first through-opening opens a pathway between the at least one first distal conduit and the first proximal conduit, and upon movement of the first piston into the second position the first through-opening opens a pathway through the at least one first distal conduit and the second proximal conduit.

The present invention relates to a valve mechanism and a method for the use of a valve mechanism for a suction and/or irrigation instrument, which comprises at least one first valve part of the kind comprising a first tubular housing provided with at least one first distal conduit for coupling to the instrument, and conduit means for communicating with a suction/irrigation source, said conduit means comprises at least a first and a second proximal conduits and a first piston movably arranged in the first tubular housing between a first position and a second position, said first piston has a first through-opening intersecting the longitudinal axis of the first piston.

Laparoscopic, endoscopic or keyhole surgery is a minimally invasive surgical procedure, where operations in the abdomen are performed using surgical tools which are usually inserted through body cavity walls via trocar valves, inserted into small incisions in the body cavity walls. The key laparoscopic surgical tool is the laparoscope, which usually is a small diameter telescopic or fiberoptic lens connected to a video camera and/or a light source, which allows a surgeon to monitor the abdominal surgical site via a visual monitor.

The surgical procedure is performed through at least two small incisions where the laparoscope is inserted into the abdominal cavity through one hole, and additional surgical tools inserted through at least one other small incision. Through these incisions it is possible to perform a number of different surgical operations within the abdominal cavity, without many of the complications that follow open cavity surgeries. As the abdominal cavity is practically closed during the operation, it is important to be capable of disposing the waste material, such as blood, fluids, tissue fragments, etc., within the abdominal cavity using a tool that may access the abdominal cavity through the trocar valves.

Such a tool is known from EP 0 537 573 A2, which describes a suction and irrigation tool which may be inserted into a small incision and provide suction through a small diameter cannula from the surgical site in the abdominal cavity. This tool may also be used to provide irrigation along with the suction in the abdominal cavity, to clean the surgical site of waste material resulting from the surgical procedure. A major disadvantage with the described tool is that the valves for the mechanical actuation of the suction or irrigation have ports on the suction channel, which is connected to the small diameter cannula and the suction connection port. This means that if one of the valves is opened while there is some waste material in the suction channel, the waste material may be pressed or pulled into the valve housing, causing damage to the seals or mechanism of the valve.

Another form for suction or irrigation tool is presented in WO 2004/075715 which describes a arthroscopic suction and irrigation handpiece having one valve for suction and one valve for irrigation. The valves of this handpiece are trumpet valves having a threaded opening on the valve housing for providing suction and irrigation means to the handpiece. This means that the vacuum force providing the suction means, has to be transferred through two 90 degree bends in order to transmit the force from the suction cannula to the suction tube, which is connected to the socket opening on the valve housing. Thus, the waste material from the surgical site has to travel through the two 90 degree bends, which increases the probability of creating blocks in the suction channel.

Therefore, there is a need for a valve mechanism for a suction and/or irrigation instrument, which minimizes the risk of blockage in the suction channel.

In a first aspect according to the present invention is provided a valve mechanism of the kind mentioned in the opening paragraphs, which has a reduced tendency to block.

In a second aspect according to the present invention is provided a valve mechanism of the kind mentioned in the opening paragraphs, which provide for individual selecting between suction and irrigation.

In a third aspect according to the present invention is provided a valve mechanism of the kind mentioned in the opening paragraphs, which allows an ergonomic use in a surgical procedure.

The novel and unique feature whereby this is achieved according to the present invention is the fact that upon movement of the first piston into the first position, the first through-opening opens a pathway between the at least one first distal conduit and the first proximal conduit, and upon movement of the first piston into the second position the first through-opening opens a pathway through the at least one first distal conduit and the second proximal conduit.

The first piston operates as a manual control mechanism that controls the opening of the pathway between the at least one distal conduit and the first or the second proximal conduits, via the first through-opening. When the pathway between the first distal conduit and the first proximal conduit is open, the pathway between the first distal conduit and the second proximal conduit is closed, and vice versa. This means that the positioning of the first piston between a first and a second position controls the communication with the suction/irrigation source via flow through the first or the second proximal conduit. For example if an irrigation source is in communication with the first proximal conduit and the suction source is in communication with the second proximal conduit, the at least first distal conduit provides irrigation when the first piston is in a first position and provides suction when the first piston is in the second position.

In a preferred embodiment of the present invention the conduit means may further comprise a second tubular housing inserted to communicate with the first tubular housing via any of the at least first or second proximal conduits, a second piston which has a second through-opening intersecting the longitudinal axis of the second piston, where said second piston is movably arranged in the second tubular housing, so that in a first position the piston closes a pathway between the suction and irrigation source and the first tubular housing via the first or the second proximal conduit, and in a second position the second thorough-opening opens a pathway between the suction/irrigation source and the first tubular housing via the first or the second proximal conduit.

The above mentioned flow through the first tubular housing is in such a way that one of the pathways between the first distal conduit and the first or the second proximal conduit is perpetually open, i.e. the valve mechanism is either arranged for suction or irrigation. The second tubular housing, having a second piston having a second through-opening, is arranged to control the flow through one of the first or second proximal conduits, such that when the second piston is in a first position there is no flow through any one of the proximal conduits. For example in the case where the second tubular housing arranged to communicate with the first tubular housing via the first proximal conduit, which is in communication with the irrigation source, the first piston in a first position would open the pathway to the first distal conduit, and the second piston in the first position would close the communication with the irrigation source. This arrangement is designed to close both pathways to the first distal conduit, and the valve mechanism is therefore in a inactive state, where there is no flow through the first distal conduit.

In order to activate the irrigation in the above mentioned example, the second piston would have to be moved into its second position, where the second through opening opens a communication pathway between the first distal conduit and the irrigation source, having the first piston in a first position. However, if the first piston is in its second position as the second piston is actuated into its second position, the first piston has opened the pathway to the second proximal conduit and therefore closed the pathway between the first distal conduit and the first proximal conduit.

In the embodiments of the present invention the first proximal conduit may provide means for irrigation and the second proximal conduit provide means for suction or vice versa. In this way, a single one proximal conduit provides communication means to either the suction or the irrigation source, where the choice of which is not crucial for the operation of the valve mechanism of the present invention.

In a preferred embodiment of the present invention the longitudinal axis of the first proximal conduit is displaced from the longitudinal axis of the first distal conduit. By displacing the first proximal conduit away from the first distal conduit, the opening into the second proximal conduit may be aligned opposite the opening into the first distal conduit, on the opposite side of the first tubular housing. Therefore, when the first piston is in a second position the first through-opening is level to the openings into the first distal conduit and the second proximal conduit and the flow between the conduit openings is along the shortest possible pathway. In this case, if the second proximal conduit provides communication to the suction means, the flow between the conduits would not have to travel through any corners, angles or bends through the first tubular housing, minimizing the risk of forming a blockage in the pathway.

In the case where the second tubular housing is inserted to communicate with the first tubular housing via the first proximal conduit and the longitudinal axis of the first proximal conduit is displaced as mentioned above, the longitudinal axis of the second proximal conduit may diverge from the longitudinal axis of the first proximal conduit, at an angle which is larger than 0° and smaller than 90°, preferably between 10° and 30°, more preferrably between 15° and 25°, most preferred app. 20°.

If the first and the second proximal conduits were positioned in parallel positions side by side, the second tubular housing would have to open into the second proximal conduit as the second piston would intersect the second proximal conduit when actuated into its second position. To avoid this, the second proximal conduit is positioned such that it diverges from the first proximal conduit at an angle, and the second tubular housing is positioned at a position on the first proximal conduit where the distance between the first proximal conduit and the second proximal conduit is substantially equal to or larger than the movement of the piston when moving from its first position to the second position. This means that the second piston does not intersect the second proximal conduit when positioned in its second position.

In a preferred embodiment of the present invention, the valve mechanism may have means for return movement of the respective first and/or second pistons to their respective first positions when respective first and second pistons are forced towards their respective second positions. In this preferred embodiment the valve mechanism, having the pistons in their first position, is in an inactive state and the return movement ensures that when the valve mechanism is not being operated, the flow pathways for suction and irrigation are closed.

In one embodiment of the present invention the means for return movement may be a compression spring. The compression spring is in an unloaded state when the first and/or second piston is in its first position and in a compressed, loaded state when the first and/or second piston is in its second position, ready to return the pistons to their respective first positions using the springs stored mechanical energy.

The first through-opening of the first piston of the first tubular housing is designed to open a pathway between the first distal conduit and either the first or the second proximal conduit, where in a preferred embodiment of the present invention the at least a part of the first through-opening may intersect the longitudinal axis of the first piston at least partially at an intersecting angle that is oblique to said longitudinal axis. The oblique intersecting angle allows the flow to or from the first or the second proximal conduit to be channeled in a direction towards the first distal conduit.

In the embodiments of the present invention where the first through-opening is designed to intersect the longitudinal axis at an oblique intersecting angle, the intersecting angle may be between 10°-70°, preferably between 20°-50°, and most preferred app. 35°. In an embodiment where the first or the second proximal conduit is displaced from the longitudinal axis of the first distal conduit, the intersecting angle ensures that the flow is channeled to or from the first or second proximal which is displaced from the longitudinal axis toward or from the distal conduit via the first through-opening.

In a preferred embodiment the pistons are inserted inside the tubular housings where a first sealing ring may be provided at the first piston to seal the first through-opening and optionally one or more second sealing rings may be provided upstreams and/or downstreams of the first through-opening and/or upstreams and/or downstream of the second through-opening of the second piston. These sealing rings ensure that the pistons are sealed within the tubular housings and the pathways communicating with the first or the second tubular housing are sealed from each other and the environment outside the valve mechanism, ensuring that there is a minimal or no loss of suction or irrigation forces within the tubular housings.

In one embodiment of the present invention where the opening into the second distal conduit from the first tubular housing is in line with the longitudinal axis of the first distal conduit the inner diameter of the at least first distal conduit and the second proximal conduit of the valve mechanism may be substantially equal in size, furthermore having the inner diameter of the first through-opening substantially equal in size. This means that when the second proximal conduit is used for suction, there are no constrictions along the suction pathway, which minimizes the risk of objects that have been sucked into the valve mechanism getting stuck inside the suction pathway, jamming the flow. In a preferred embodiment of the present invention, the distal conduit, the first through-opening and the second proximal conduit may have a inner diameter that is approximately 10 mm. This means that a instrument which has an inner diameter of approximately 10 mm may be coupled to the valve mechanism, allowing the suction of a relatively large particle having in a compressed state a diameter equal or less than 10 mm.

In one embodiment of the present invention where the second proximal conduit is in communication with the suction source, and a situation occurs where the suction force produced by the suction source may be too powerful for a specific suction task, a reduction in suction force might be needed. This may be achieved by arranging the first tubular housing with a vent hole, which communicates with the first through-opening when the first piston is in its second position. The vent hole may be provided with user-operated means for opening and closing the hole. When the vent hole is left open, and the first piston is in its second position, the vent hole provides a pathway for air to be sucked from the surrounding environment, which reduces the suction forces affecting the first distal conduit. When the vent hole is closed, and the first piston is in its second position, the entire suction force of the suction source is transmitted to the distal conduit.

The valve mechanism, in a preferred embodiment, is designed to be operated by the hand of the user, and in order to facilitate the hand operation the valve mechanism may be provided with a palm rest part, arranged to fit in the palm of a hand. Furthermore, for providing the valve mechanism with actuating means for moving the first and/or the second piston into their first and/or second position, the first free end of the first piston and/or a second free end of the second piston may each have their respective pressure face. Using the palm rest part and the pressure faces of the pistons, the valve mechanism may be held and operated single handedly.

In a preferred embodiment, the valve mechanism may be symmetrical across a plane which intersects and is parallel to the longitudinal axis of the first piston, the longitudinal axis of the at least first distal conduit and the longitudinal axis of the at least first and second proximal conduits, arranging both right and left hand operation. This means that all functions of the valve mechanism may be equally easily reached from either side of the valve mechanism.

As the valve mechanism is intended for hand operation it is advantageous that the valve mechanism may easily be maneuvered and moved into the desired position. Thus, in an advantageous embodiment of the present invention the suction and/or irrigation source may be connected to the valve mechanism using flexible tubing means via connectors provided on the first and/or the second proximal tubular conduits.

In another advantageous embodiment the flexible tubing means may be of partial or full spiral tubular material. Spiral tubular material is a very flexible type of tubular material, where the spiral part of the material is strong and the intermediate material is weak, compared to a uniform tubular material. The spiral part provides strength in the radial direction, such that the tubing material retains its shape under positive or negative pressure. The intermediate material, on the other hand gives the spiral tubular material its increased flexibility, compared to uniform tubular material, as the weakened sections are designed to easily compress or expand without compromising the flow pathway.

The spiral tubular material may be used to connect the suction or irrigation source to the valve mechanism, where the spiral tubular material may comprise the entire flexible tubing or a part thereof, whichever embodiment is more advantageous for a specific situation. The spiral tubing increases the maneuverability of the valve mechanism, as the spiral tubular material allows bending and flexing using low forces.

In a preferred embodiment of the present invention, the suction and/or irrigation instrument may be a surgical instrument. The surgical instrument may for example be a laparoscopic elongated stiff tubular structure which may be entered into the body using a small incision or via a trocar valve, or a stiff tubular structure for conventional open surgery, where suction and irrigation tasks may be performed, using the valve mechanism, within the bodily cavities. The suction and irrigation tasks may be performed through the same elongated tubular structure, as the valve mechanism ensures that only one functionality may be accessed at a single time.

Furthermore, within the scope of the present invention the preferred use of the valve mechanism is for use with a surgical instrument, however the valve mechanism may also be used for other purposes, such as along with other types of suction and/or irrigation instruments which are designed for other purposes. The instrument may be an elongated stiff tubular structure which may be used for cleaning surface areas, such as a ships deck, any kind of floor material and similar, where the irrigation provides uncontaminated fluids for cleaning the surface area and the suction is used to remove the contaminated fluids from the surface area.

The present invention also provides a suction and/or irrigation method where the method comprises the steps of providing a valve mechanism according to the present invention and displacing the first and/or the second piston to provide fluid communication with the irrigation source through the first proximal conduit and/or to provide fluid communication with the suction source through the second proximal conduit. In this way a method is provided which is simple and makes the valve mechanism very simple to use.

In a preferred embodiment of the method according to the present invention the steps of displacing the first and/or the second piston may comprise any of the steps a)-d) in an arbitrary order: a) displacing the first and second pistons into their respective first positions to close the pathway between the distal conduit and the suction and irrigation source keeping the valve mechanism in an inactive state, b) displacing the first piston into the second position and displacing the second piston into the first position to open a pathway between the distal conduit and the suction source, keeping the valve mechanism in a suction state, c) displacing the first piston into the first position and the second piston into the second position to open a pathway between the distal conduit and the irrigation source, keeping the valve mechanism in a irrigation state, and d) displacing the first and second piston into their respective second positions to open the pathway between the distal conduit and the suction source, where the first piston closes the pathway between the distal conduit and the irrigation source, keeping the valve mechanism in a suction state.

The displacement of the first or the second piston into a first or a second position may be done in a gradual manner, where the through-openings of the pistons may gradually open the pathways, as the through-openings increasingly open into the first or the second proximal conduit. This means that the suction and/or irrigation forces may be controlled by the positioning of the first or the second piston, where the pistons either in the first position or the second position fully open the pathways.

The invention will be explained in greater detail below where further advantageous properties and example embodiments are described with reference to the drawings, in which

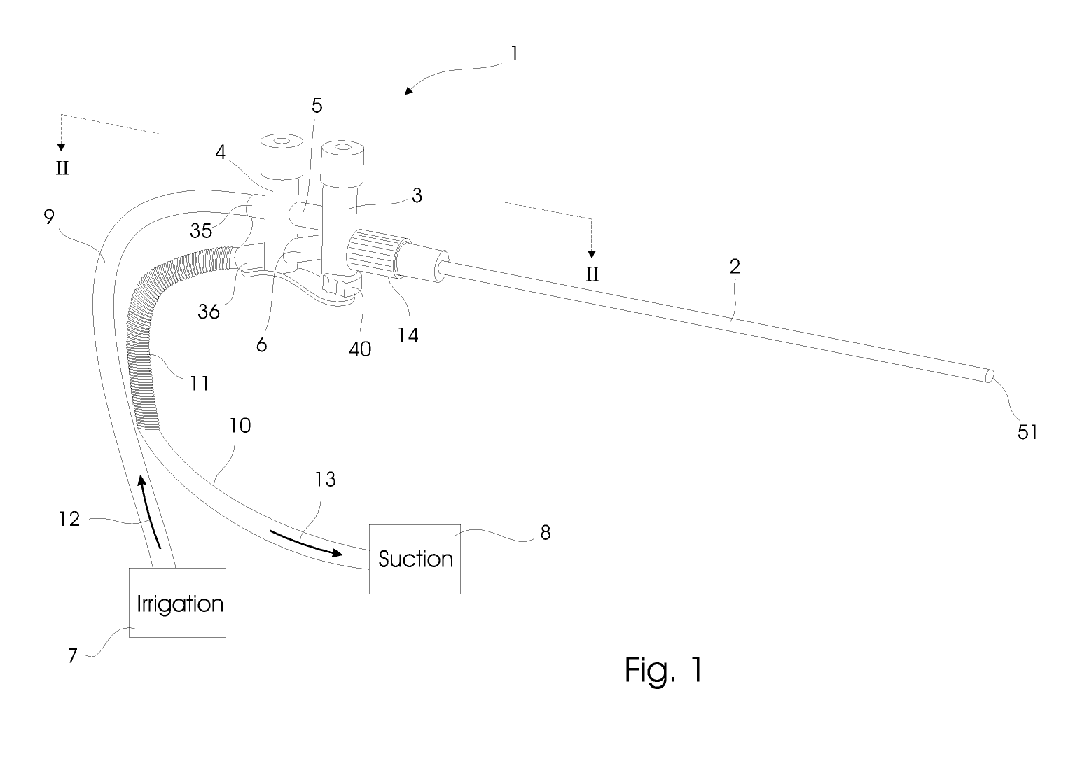

FIG. 1 shows a perspective view of a valve mechanism for suction and/or irrigation,

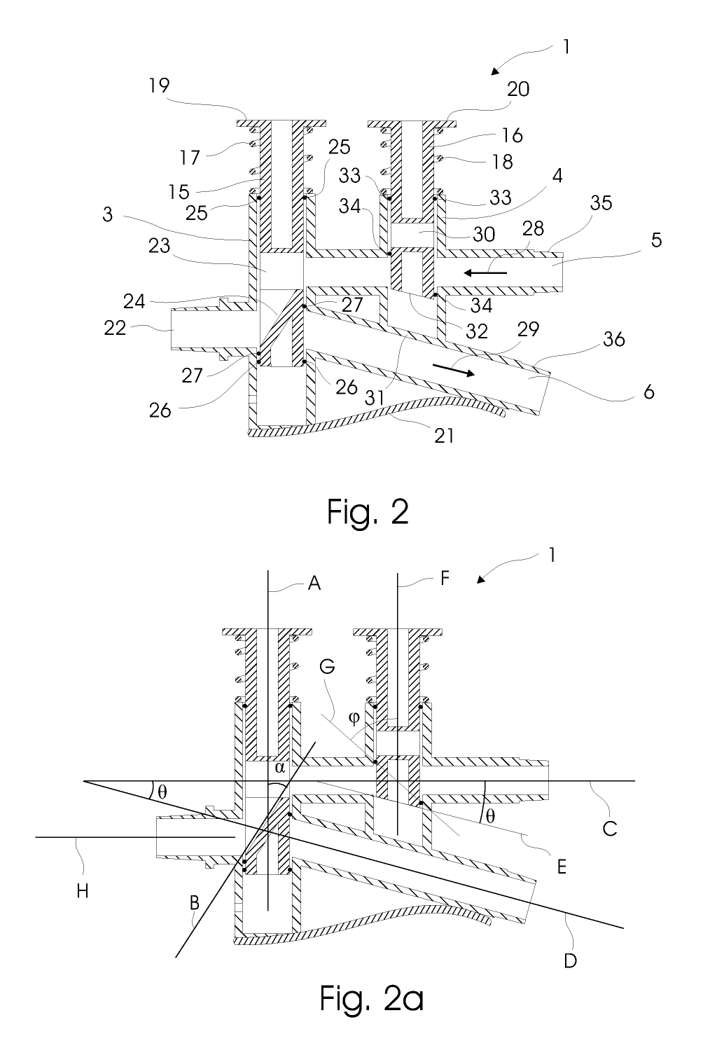

FIG. 2 shows a sectional view of the valve mechanism taken along line III-III in FIG. 1, wherein the first and second pistons are in their respective first position, arranging the valve mechanism in an inactive state,

FIG. 2a shows the same, where the axis and angles of the valve mechanism are shown in detail,

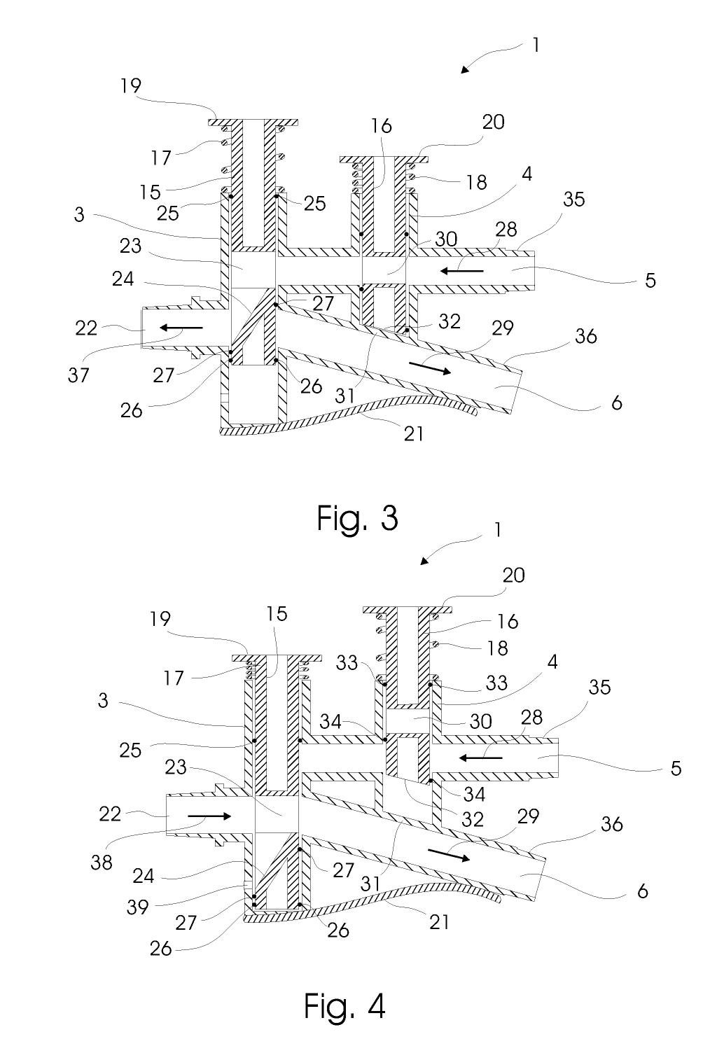

FIG. 3 shows the same but with the first and second pistons in their first and second position respectively, arranging the valve mechanism in an irrigation state,

FIG. 4 shows the same but with the first and second pistons in their second and first position respectively, arranging the valve mechanism in a suction state,

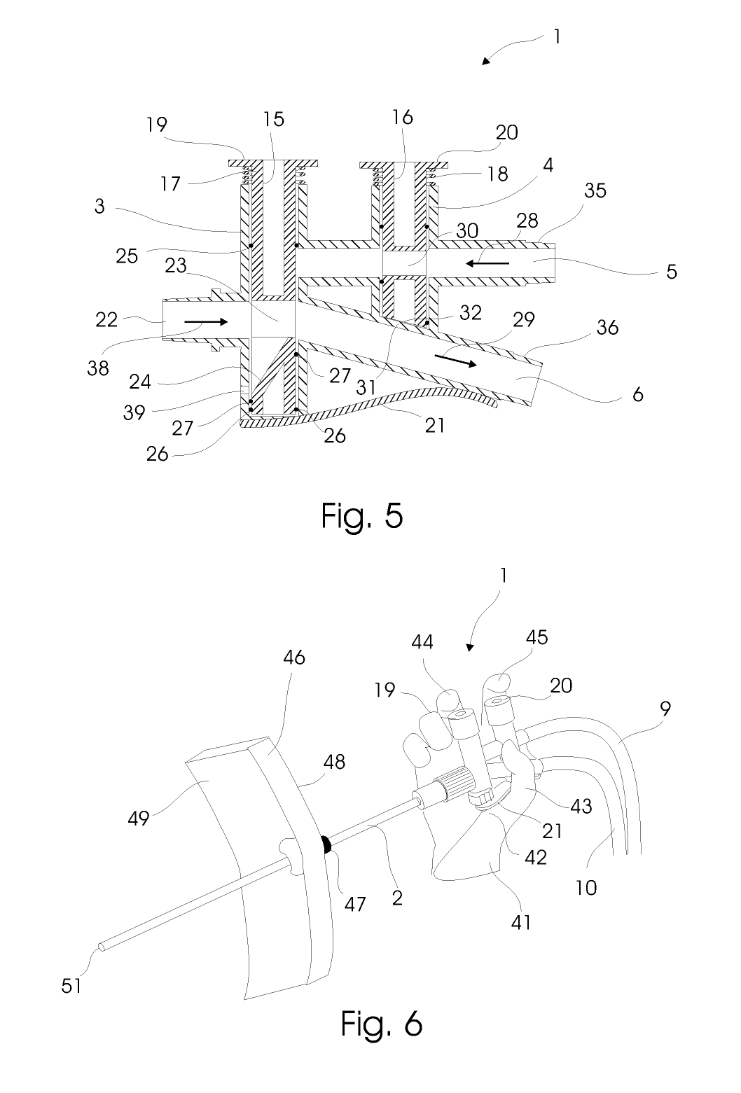

FIG. 5 shows the same but with the first and second pistons in their respective second position, arranging the valve mechanism in a suction state, and

FIG. 6 shows the same operated by a surgeon in a laparoscopic procedure.

FIG. 1 shows a perspective view of a preferred embodiment of a valve mechanism 1 for suction and/or irrigation according to the present invention. The valve mechanism 1, which is connected to a stiff elongated tubular structure 2, has a first tubular housing 3, which via a first proximal conduit 5 and a second proximal conduit 6 are arranged to communicate with a second tubular housing 4. The second tubular housing 4 is connected to a irrigation source 7 via a flexible tubing for irrigation 9 and to a suction source 8 via a flexible tubing for suction 10. The irrigation source 7 provides fluid supply via the first proximal conduit 5 between the housings 3,4 to the instrument 2 in a direction indicated with arrow 12, while the suction source 8 provides suction through the instrument 2 via the second proximal conduit 6 and the housings 3,4 in a direction indicated with arrow 13.

In this embodiment of the present invention the suction tubing 10 is partially made of a spiral tubing material 11, which provides increased flexibility when bending and flexing of the suction tubing 10 is necessary. It is obvious to the skilled person that the suction tubing may just as well be fully made of spiral tubing, that the irrigation tubing 9 may also be made of spiral tubing or that neither the irrigation 9 or suction tubing 10 are made of spiral tubing, depending on the intended use of the valve mechanism 1.

The stiff elongated tubular structure 2 is connected to the valve mechanism 1 by means of coupling means 14 for maintaining a secure connection between the tubular structure 2 and the valve mechanism 1. The stiff elongated tubular structure 2 has an opening 51 in the distal end of the tubular structure 2 which may be brought into contact with the fluid to be sucked away or which is arranged in relation to the site to be irrigated.

FIG. 2-FIG. 5 show a series of sectional views of the valve mechanism 1 along the line II-II in FIG. 1, having a first piston 15 positioned inside the first tubular housing 3, and a second piston 16 positioned inside the second tubular housing 4 respectively, in their respective first position and/or second positions. The same reference numerals are used for like parts in FIG. 2-FIG. 5. Furthermore, for the sake of clarity and conciseness FIG. 2a is separately used to show the axis and angles mentioned in the following description regarding FIG. 2-FIG. 5.

FIG. 2 shows the valve mechanism 1 in an inactive state where the first 15 piston and the second piston 16 are in their respective first positions, arranging the valve mechanism 1 in an inactive state. The first piston 15 and the second piston 16 are kept in their respective first position using a first compression spring 17 and a second compression spring 18, that maintain a spring tension in the unloaded position between the tubular housings 3,4 and the opposing side of the first pressure face 19 and the second pressure face 20, which are arranged at the free ends of the pistons 15,16, respectively. In the following when using the terms top and/or bottom in relation to the valve mechanism 1 the top is the side of the valve mechanism 1 that has the pressure faces 19,20, while the bottom is the face that is opposite to the pressure faces 19,20.

On the bottom side of the valve mechanism 1, the valve mechanism 1 has a palm rest 21, which provides an ergonomic surface to fit in the palm of the hand when the valve mechanism is used and controlled single handedly. The first tubular housing 3 has a distal conduit 22, which can be brought in fluid communication with the first proximal conduit 5 or the second proximal conduit 6 by displacing the first piston 15 inside the first tubular housing 3. In this embodiment of the present invention the first proximal conduit 5 is positioned such that its longitudinal axis C is displaced from the longitudinal axis H of the distal conduit 22, as shown in FIG. 2a.

The first piston 15, having a longitudinal axis A, has a through-going opening 23 where a portion of the opening 23 has an oblique wall 24 sloping along an axis B at an angle α to the longitudinal axis A, as shown in FIG. 2a, which opens a pathway between the distal conduit 22 and the first proximal conduit 5. In this embodiment the angle α is approximately 35°. The first piston 15 is provided with at least three sealing rings, a first sealing ring 25 and a second sealing ring 26 which are positioned perpendicular to the longitudinal axis A of the piston 15 which provides sealing means between the piston 15 and the housing 3 where the first sealing ring 25 is positioned close to the top of the tubular housing 3 and the second 27 is placed at the bottom end of the piston 15. The third sealing ring 27 is positioned around the first piston at an oblique angle to the longitudinal axis A that is substantially equal in size to the angle α between axis A and B of FIG. 2a, as abovementioned. When the first piston is in a first position, the third sealing ring 27 ensures that there are no leakages between the first proximal conduit 5 and the second proximal conduit 6.

The direction of the irrigation flow in the first proximal conduit 5 is in the direction indicated by the arrow 28, while the direction of the suction flow in the second proximal conduit 6 is in the direction indicated by the arrow 29.

The second piston 16 arranged in the second tubular housing 4 has a through-going opening 30 which in the piston's 16 first position closes the flow through the first proximal conduit 5 from the irrigation source to the distal conduit 22. The closing occurs as a result of that the opening 30 is displaced away from the first proximal conduit 5, in the second piston's 16 first position. In this particular embodiment the second tubular housing 4 is only in fluid communication with the first proximal conduit 5, and the lowest part of the second tubular housing 3 is an end wall 31 which separates the second tubular housing 3 and the second proximal conduit 6.

The longitudinal axis C of the first proximal conduit 5 and the longitudinal axis D of the second proximal conduit, diverge at an angle θ, as shown in FIG. 2a, which in this embodiment is approximately 20°. The end wall 31, separating the second tubular housing 3 and the second proximal conduit 6 is parallel to the longitudinal axis D, of the second proximal conduit 6, as shown in FIG. 2 along with FIG. 2a. As a result, the bottom end part 32 of the second piston 16 is formed at an angle θ between axis C and axis E, which is equal to the angle θ between axis C and D, and is thus parallel to the end wall 31 of the second tubular housing 4, such that the end wall 31 does not obstruct the movement of the second piston 16 into its second position, as shown in FIG. 3.

The second piston 16 is provided with at least two sealing rings 33,34, providing sealing means between the tubular housing 4 and the piston 16. The first sealing ring 33 is positioned around the second piston 16 close to the top of the tubular housing 4, having the piston 16 in its first position, where the sealing ring 33 is perpendicular to the longitudinal axis F, shown in FIG. 2a, of the second piston 16. The second sealing ring 34 is positioned close to the bottom end 32 of the piston 16 at an angle φ between the longitudinal axis F of the second piston and the axis G of the second sealing ring 34, as shown in FIG. 2a, where the sealing ring 34 prevents leakage from the first proximal conduit 6 towards the distal conduit 22 when the second piston 16 is in its first position. The angle φ, shown in FIG. 2a, is preferably between 20° and 70°, more preferably between 30 and 60, and most preferred approximately 40° in this preferred embodiment.

The second tubular housing 4 of the valve mechanism 1 is provided with connection means 35,36 for connecting flexible tubings 9,10 to provide fluid communication with a suction and/or irrigation source, such as seen in FIG. 1.

FIG. 3 shows the valve mechanism 1 in an irrigation state, where the second piston 16 has been displaced into its second position, bringing the second through-opening 30 in line with the first proximal conduit 5. The through-opening 30 opens a pathway from the suction source via the proximal conduit 5 towards the distal conduit 22, providing an irrigation fluid flow through the distal conduit 22 in the direction indicated by the arrow 37. The first piston 15, still in its first position, opens the pathway between the first proximal conduit 5 and the distal conduit 22, closing the pathway between the second proximal conduit 6 and the distal conduit 22.

FIG. 4 shows the valve mechanism 1 in a suction state, where the first piston 15 has been displaced into its second position, and the second piston 16 is in its first position. In the first piston 15's first position the first through-opening 23 is in line with the distal conduit 22 and the second proximal conduit 6, opening the pathway between said conduits. The suction forces transferred through the second proximal conduit 6 provide a suction fluid flow through the distal conduit 22 in the direction indicated by the arrow 38.

As the first piston 15 is positioned in its second position, the oblique sloping wall 24 of the through-opening 23 is in fluid communication with a vent hole 39 in the first tubular housing 2. In a preferred embodiment the vent hole 50 is closeable, using a slideable cover 40 in FIG. 1, and when the vent hole 39 is open the suction forces affecting the distal conduit 22 are reduced, while when the vent hole 39 is closed the suction forces affecting the distal conduit 22 are substantially of the same magnitude as the suction forces provided in the second proximal conduit 6.

FIG. 5 shows the valve mechanism 1 in a suction state, where the first piston 15 is in its second position and the second piston 16 is in its second position. The first piston 15, blocks the pathway between the first proximal conduit 5 and the distal conduit 22, such that the distal conduit 22 is only affected by the suction forces from the second distal conduit 6, maintaining a flow in the direction of the arrow 38.

FIG. 6 shows a perspective view of a valve mechanism 1 for suction and/or irrigation. The valve mechanism 1 is equipped with a stiff elongated tubular structure 2 for use in a laparoscopic suction and irrigation procedure. The valve mechanism 1 of the assembled instrument is placed in the hand 41 of e.g. a surgeon so that the palm rest 21 of the valve mechanism 1 lies in the palm 42 of the surgeons hand 41. The valve mechanism 1 may be held in place using the thumb 43 of the hand 41, where the first pressure face 19 and the second 19 pressure face may be displaced using the middle 44 and the index finger 45, respectively.

The laparoscopic suction and irrigation instrument 2 is inserted through a trocar valve 47 inserted into a body cavity wall 46, where the valve mechanism 1 may be maneuvered outside the body, i.e. the external surface 48 of the wall 46 while the opening 51 of the instrument 2 is inserted into a position which is on the same side as the inner surface 49 of the wall. In this position the valve mechanism 1 may be used to provide suction and/or irrigation within a body cavity, where the surgeon uses his fingers 44,45 for controlling the valve mechanism 1, and his hand for positioning the opening 51 of the instrument 2.

The valve mechanism 1 along with the laparoscopic instrument 2 may be used to remove coagulated blood, bodily fluids, tissue fragments, puss and other kinds of excess material which has to be removed from a operation site. The irrigation may be used to ensure that the operation site is free from debris and unwanted material. If a specific fragment of a material is too large for the valve mechanism to remove, the material may be crushed or split apart using an electrosurgical or mechanical surgical instrument, such that the size is suitable for the valve mechanism to remove.

The valve mechanism 1 of the present invention may be constructed mainly from plastic materials which makes the valve mechanism cheap to produce as most of the material may be molded. The skilled person would recognize that some parts of the valve mechanism are not optimally molded, such as the compression springs and possibly the sealing rings.

The plastic parts are relatively cheap in manufacturing, the valve mechanism may preferably be a single use instrument, made out of environmentally friendly material, such that a used instrument may be recycled as is well known within the art of recycling. Furthermore, the valve mechanism may be constructed as a reusable instrument, where all the different parts of the valve may be removed and cleaned in a sterile manner, such that when reused, the valve mechanism withstands the cleanliness level required for surgery.

The plastic parts of the valve mechanism may very well be constructed in transparent material, allowing the operator to see if there are any blockages within the valve mechanism caused by large particles or material being sucked.

It is obvious to the person skilled in the arts that any kind of surgical instrument suitable for suction or irrigation could be coupled to the valve mechanism.

1-20. (canceled)

21. A valve mechanism for an instrument that includes suction, irrigation or both suction and irrigation, including at least one first valve part comprising:
a first tubular housing provided with at least one first distal conduit for coupling to the instrument, and conduit means for communicating with a suction or irrigation source, with the conduit means comprising at least first and second proximal conduits, and
a first piston movably arranged in the first tubular housing between a first position and a second position, with the first piston has a first through-opening intersecting the longitudinal axis (A) of the first piston;
wherein, upon movement of the first piston into the first position, the first through-opening opens a pathway between the at least one first distal conduit and the first proximal conduit, and upon movement of the first piston into the second position the first through-opening opens a pathway through the at least one first distal conduit and the second proximal conduit.

22. The valve mechanism according to claim 21, wherein the conduit means further comprises:
a second tubular housing inserted to communicate with the first tubular housing via any of the at least first or second proximal conduits,
a second piston which has a second through-opening intersecting the longitudinal axis (F) of the second piston,
wherein the second piston is movably arranged in the second tubular housing, so that in a first position the piston closes a pathway between the suction or irrigation source and the first tubular housing via the first or second proximal conduit, and in a second position the second thorough-opening opens a pathway between the suction and irrigation source and the first tubular housing via the first or the second proximal conduit.

23. The valve mechanism according to claim 21, wherein one of the first or second proximal conduits provides irrigation and the other provides suction.

24. The valve mechanism according to claim 21, wherein the first proximal conduit has longitudinal axis (C), the first distal conduit has longitudinal axis (H) and longitudinal axis (C) is displaced from longitudinal axis (H).

25. The valve mechanism according to claim 24, wherein the second proximal conduit has longitudinal axis (D) which diverges from longitudinal axis (C) of the first proximal conduit at an angle (θ) which is larger than 0°-15° and smaller than 25°-90°.

26. The valve mechanism according to claim 21, wherein one of the first or second tubular housings, or both, include means for return movement of the respective first or second pistons to their respective first positions after the respective first and second pistons are forced towards their respective second positions.

27. The valve mechanism according to claim 26, wherein the means for return movement is a compression spring.

28. The valve mechanism according to claim 21, wherein at least part of the first through-opening intersects the longitudinal axis (A) of the first piston at least partially at an intersecting angle (α) that is oblique to longitudinal axis (A).

29. The valve mechanism according to claim 28, wherein the intersecting angle (α) is between 10°-20° and 50°-70°.

30. The valve mechanism according to claim 21, which further comprises a first sealing ring at the first piston to seal the first through-opening and optionally one or more second sealing rings are provided upstream, downstream or both upstream and downstream of the first through-opening or upstream, downstream or both upstream and downstream of the second through-opening of the second piston.

31. The valve mechanism according to claim 21, wherein the at least first distal conduit and the second proximal conduit of the valve mechanism each have inner diameters that are substantially equal in size.

32. The valve mechanism according to claim 21, wherein the first tubular housing comprises a vent hole communicating with the first through-opening when the first piston is in its second position.

33. The valve mechanism according to claim 21, wherein the valve mechanism is provided with a palm rest part arranged to fit in the palm of a hand.

34. The valve mechanism according to claim 24, wherein one of a first free end of the first piston or a second free end of the second piston each has a respective pressure face.

35. The valve mechanism according to claim 21, wherein the valve mechanism is symmetrical across a plane which intersects and is parallel to the longitudinal axis (A) of the first piston, as well as a longitudinal axis (H) of the at least first distal conduit and longitudinal axes (C,D) of the at least first and second proximal conduits.

36. The valve mechanism according to claim 21, wherein the suction or irrigation source or both are connected to the valve mechanism using flexible tubing means via connectors provided on the first or second proximal tubular conduits.

37. The valve mechanism according to claim 24, wherein the flexible tubing means are of partial or full spiral tubular material.

38. The valve mechanism according to claim 24, wherein the suction or irrigation instrument is a surgical instrument.

39. A suction or irrigation method which comprises:
providing a valve mechanism according to claim 22,
displacing the first or second piston to provide fluid communication with the irrigation source through the first proximal conduit or to provide fluid communication with the suction source through the second proximal conduit.

40. The method according to claim 39, wherein the displacing the first or the second piston comprises:
displacing the first and second pistons into their respective first positions to close the pathway between the distal conduit and the suction and irrigation source keeping the valve mechanism in an inactive state;
displacing the first piston into the second position and displacing the second piston into the first position to open a pathway between the distal conduit and the suction source, keeping the valve mechanism in a suction state;
displacing the first piston into the first position and the second piston into the second position to open a pathway between the distal conduit and the irrigation source, keeping the valve mechanism in a irrigation state; and
displacing the first and second pistons into their respective second positions to open the pathway between the distal conduit and the suction source, where the first piston closes the pathway between the distal conduit (22) and the irrigation source (7), keeping the valve mechanism in a suction state.
